(19) United States
(12) Patent Application Publication
(10) Pub. No.: US 2016/0032234 A1
(43) Pub. Date: Feb. 4, 2016

(54) METHODS AND DEVICES FOR MECHANICAL AND ELECTRICAL STIMULATION OF STEM CELL MONOLAYER AND 3D CULTURES FOR TISSUE ENGINEERING APPLICATIONS

(71) Applicants: Francesc Xavier ROSELL FERRER, Barcelona (ES); Benjamín SÁNCHEZ TERRONES, Barcelona (ES); Ramón BRAGÓS BARDIA, Barcelona (ES); Antonio BAYÉS GENÍS, Barcelona (ES); Aida LLUCIÀ VALLDEPERAS, Barcelona (ES)

(72) Inventors: Francesc Xavier ROSELL FERRER, Barcelona (ES); Benjamín SÁNCHEZ TERRONES, Barcelona (ES); Ramón BRAGÓS BARDIA, Barcelona (ES); Antonio BAYÉS GENÍS, Barcelona (ES); Aida LLUCIÀ VALLDEPERAS, Barcelona (ES)

(73) Assignees: UNIVERSITAT POLITÈCNICA DE CATALUNYA, Barcelona (ES); FUNDACIÓ INSTITUT D'INVESTIGACIÓ EN CIÈNCIES DE LA SALUT GERMANS TRIAS I PUJOL, Badalona (ES)

(21) Appl. No.: 14/407,728

(22) PCT Filed: Jun. 13, 2012
(86) PCT No.: PCT/EP2012/061224

§ 371 Date: Oct. 5, 2015

(51) Int. Cl. C12M 1/42; C12M 1/00; C12M 1/34

(52) U.S. Cl. CPC C12M 35/02; C12M 35/06; C12M 41/00; C12M 35/04; C12M 23/26

(57) ABSTRACT

Devices and methods for stimulating mechanically or electromechanically stem cell monolayer and 3D cultures for tissue engineering applications are disclosed. A bracket is proposed to be used in a disposable stimulation device. A disposable stimulation device is also proposed for accommodating a cell culture in a flexible area. Finally, an apparatus is proposed to mechanically extend the disposable stimulation device to induce mechanical stimulation. Additionally, a pair of electrodes placed at two opposing sides of the flexible area creates an electric field to induce electrical stimulation. Accordingly, a method to stimulate electromechanically a cell culture is proposed.

The present invention relates to devices and methods for stimulating cell cultures. More particularly, the present invention relates to devices and methods for electromechanical stimulation of cell cultures of stem cell monolayer and 3D cultures for tissue engineering applications.

### BACKGROUND ART

Nowadays, cardiovascular diseases have a huge impact on population health, becoming the first cause of death in the developed world. Heart failure is the end-stage of many cardiovascular diseases, but the leading cause is the presence of a large scar due to an acute myocardial infarction. Acute myocardial infarction normally happens when blood supply to the heart is interrupted. Therapeutic strategies that limit adverse post-ischemic remodelling in heart failure may prevent ventricular dilatation and maintain the structural support necessary for effective cardiomyocyte contraction. Current treatments under development consist in cellular cardiomyoplasty, where myocardial or stem cells are encapsulated in natural or artificial scaffolds (collagens, polymeric fibers, respectively) and grafted onto infarcted ventricles with the hope that cells will contribute to the generation of new myocardial tissue (Vunjak-Novakovic, G. et al., 2010. Challenges in Cardiac Tissue Engineering. TISSUE ENGINEERING: Part B, 16(2), pp.169-187). This approach seems to have a beneficial effect although it is not well develop yet because most of the implanted cells die soon after treatment and recovery is only modest (Genovese J et al. Cell based approaches for myocardial regeneration and artificial myocardium. Curr Stem Cell Res Ther. 2: 121-7, 2007; Patel A N, Genovese J A. Stem cell therapy for the treatment of heart failure. Curr Opin Cardiol. 22: 464-70, 2007; Chachques J C, Trainini J C, Lago N, Cortes-Morichetti M, Schussler O, Carpentier A. Myocardial Assistance by Grafting a New Bioartificial Upgraded Myocardium (MAGNUM trial): clinical feasibility study. Ann Thorac Surg. Mar;85 (3):901-8, 2008; Cortes-Morichetti M, Frati G, Schussler O, Van Huyen J P, Lauret E, Genovese J A, Carpentier A F, Chachques J C. Association between a cell-seeded collagen matrix and cellular cardiomyoplasty for myocardial support and regeneration. Tissue Eng. Nov;13(11):2681-7, 2007).

Moreover, the cell type with the best ability to restore cardiac tissue remains elusive. Despite the identification of resident cardiac stem cells (Beltrami A P, Barlucchi L, Torella D, Baker M, Limana F, Chimenti S, et al. Adult cardiac stem cells are multipotent and support myocardial regeneration. Cell 2003; 114(6): 763-6.; Oh H, Bradfute S B, Gallardo T D, Nakamura T, Gaussin V, Mishina Y, et al. Cardiac progenitor cells from adult myocardium: homing, differentiation, and fusion after infarction. Proc Natl Acad Sci U S A 2003; 100(21): 12313-8), tissue repair after damage is deficient, and a great deal of attention has been placed on finding the best cell type to repair injured tissue (Goldstein G, Toren A, Nagler A. Human umbilical cord blood biology, transplantation and plasticity. Curr Med Chem 2006; 13(11): 1249-59; Orlic D, Kajstura J, Chimenti S, Bodine D M, Leri A, Anversa P. Transplanted adult bone marrow cells repair myocardial infarcts in mice. Ann N Y Acad Sci 2001; 938: 221-29; discussion 229-30; Pittenger M F, Martin B J. Mesenchymal stem cells and their potential as cardiac therapeutics. Circ Res 2004; 95(1): 9-20; Rangappa S, Fen C, Lee E H, Bongso A, Sim E K. Transformation of adult mesenchymal stem cells isolated from the fatty tissue into cardiomyocytes. Ann Thorac Surg 2003; 75(3): 775-9). Adult stem cells have been tested in a variety of mammalian hearts, from mice to humans, after injury. In mice, the great promise of bone marrow derived progenitors has been tempered by reports showing that their cardio regenerative potential is limited and controversial (Balsam L B, Wagers A J, Christensen J L, Kofidis T, Weissman I L, Robbins R C. Haematopoietic stem cells adopt mature haematopoietic fates in ischaemic myocardium. Nature 2004; 428(6983): 668-73; Murry C E, Soonpaa M H, Reinecke H, Nakajima H O, Rubart M, Pasumarthi K B, et al. Haematopoietic stem cells do not transdifferentiate into cardiac myocytes in myocardial infarcts. Nature 2004; 428(6983): 664-8). In humans, improvement in cardiac function was shown in early clinical studies (Assmus B, Schachinger V, Teupe C, Britten M, Lehmann R, Dobert N, et al. Transplantation of Progenitor Cells and Regeneration Enhancement in Acute Myocardial Infarction (TOPCARE-AMI). Circulation 2002; 106(24): 3009-17; Strauer B E, Brehm M, Zeus T, Kostering M, Hernandez A, Sorg R V, et al. Repair of infarcted myocardium by autologous intracoronary mononuclear bone marrow cell transplantation in humans. Circulation 2002; 106(15): 1913-8), while more recent clinical trials showed only a modest increase in cardiac function after cell delivery (Dohmann H F, Silva S A, Souza A L, Braga A M, Branco R V, Haddad A F, et al. Multicenter Double Blind Trial of Autologous Bone Marrow Mononuclear Cell Transplantation through Intracoronary Injection post Acute Myocardium Infarction a MiHeart/AMI Study. Trials 2008; 9(1): 41). Hence, the search for new types of adult stem cells capable of restoring cardiac function remains a challenge.

On the other hand, cardiodifferentiation of the selected cells before implantation is an attractive approach to obtain heart regeneration. Nevertheless, cardiogenesis is a complex process. Currently, different strategies for cardiac differentiation exist, but the majority of the studied approaches protocols only attain partial results. For example, cardiomyocyte-like cells can be achieved using demethylating agents like 5-azacitidine, which up-regulates cardiac markers such as GATA-4, Nkx2.5 and cardiac troponin I, developing beating cell clusters after embryoid-body-like structures formation (Choi et al.—2004—5-azacytidine induces cardiac differentiation of P19 embryonic stem cells). Other protocols are described in the literature, where a mixture of reagents is used to obtain beating cells from human cardiomyocyte progenitor cells (Smits et al., Human cardiomyocyte progenitor cells differentiate into functional mature cardiomyocytes: an in vitro model for studying human cardiac physiology and pathophysiology, Nat. Protocols, 2009, Vol. 4-2. p. 232-243). Additionally, co-culture of mesenchymal stem cells with neonatal rat cardiomyocytes simulates cardiac differentiation and is based on the general knowledge that cardiac environmental factors are powerful inducers of cardiomyogenic process (Fukuhara et al., Direct cell-cell interaction of cardiomyocytes is key for bone marrow stromal cells to go into cardiac lineage in vitro, The Journal of thoracic and cardiovascular surgery, 1 Jun. 2003 (volume 125 issue 6, p. 1470-1479). Finally, the most inventive and natural approach for cardiodifferentiation is mimicking the cardiac electromechanical physiology, which involves physical stimuli. Few groups are working on electrical and/or mechanical stimulation. Tandon et al. demonstrated modification of gene profile and cell elongation and alignment in concordance with the electrical field (Tandon et al.—2010—Alignment and Elongation of Human Adipose-Derived Stem Cells in Response to Direct-Current Electrical Stimulation). Moreover, cells respond to tensional forces by secreting factors or up-regulating and/or down-regulating specific genes (Freytes et al., Geometry and force control of cell function, J. Cell. Biochem. Vol. 108-5, p. 1047-1058, 2009). However, the existing devices are unable to exert electromechanical stimulation in unison.

It would be desirable to provide a device and a method in which the above drawbacks are at least partly solved with a relatively simple and cost effective way.

### SUMMARY OF THE INVENTION

The present disclosure proposes a new apparatus and method for exerting mechanical and/or electrical stimulation to a cell culture with a non-invasive and aseptic approach.

The device presented in this patent application allows combination of both electrical and mechanical stimulation either independantly or simultaneously. The use of magnets allows performing mechanical stimulation with a non-invasive and aseptic novel approach.

In a first aspect, a bracket is proposed attachable to a flexible cell culture pool in order to form a stimulation device. A first portion of the bracket is adapted to accommodate a ferromagnetic element, such as a magnet or made of any other ferromagnetic material, wherein a first side of the first portion of the bracket is attachable to a side of the flexible cell culture pool. The ferromagnetic element is embedded in the first portion so that the whole bracket can be sterilizable, irrespective of the material of the embedded ferromagnetic element. The first portion of the bracket is substantially rectangular. However, a side of the first portion, opposite to the first side, may be curved so that it osculates with an internal surface of a Petri dish, as will be discussed below.

In a preferred embodiment, the bracket further includes a second portion that is adjacent to a second side of the first portion, wherein the second side shares an edge with the first side, and wherein the area of the second portion completely covers the area of the second side and even extends beyond the area of the second side of the first portion towards the common edge between the first side and the second side of the first portion.

In some embodiments the second portion is attached to the first portion and in other embodiments the second portion forms one body with the first portion. Both the first and the second portions of the bracket are made of biocompatible material, such as polydimethylsiloxane (PDMS).

In some embodiments, an adhesive layer is placed on the first side of the first portion of the bracket for attaching the bracket to the side of the flexible cell culture pool.

In a second aspect, the present invention provides a stimulation device, simply reffered to as “device”, for stimulation of cell cultures comprising at least one bracket attached to a flexible cell culture pool. In one embodiment the flexible cell culture pool comprises a flexible rectangular body. The flexible rectangular body comprises a flexible substrate encompassed by a flexible structure to form a flexible cell culture pool adapted to host a cell culture. Such an arrangement is suitable for cell cultures of the stem cell monolayer culture type. Furthermore, the at least one bracket is attached to a side of the rectangular body. More particularly, the first side of the at least one bracket is attached to one side of the rectangular flexible cell culture pool.

In a preferred embodiment a stimulation device comprises two brackets so that the the first side of the first portion of the of the first bracket is attached to one side of the rectangular flexible cell culture pool and the first side of the first portion of the second bracket is attached to a second side of the flexible cell culture pool so that the first bracket and the second bracket are placed one diametrically opposite to the other with respect to the flexible cell culture pool.

Each of the brackets comprises a first portion extending sideways and elevated with respect to the flexible cell culture pool, and is adapted to accommodate an embedded ferromagnetic element and a second portion adjacent to the first lateral portion on its upper side and extending beyond the area of the first lateral portion so as to form a recess between the flexible structure and the second portion.

In some embodiments the first portion of the bracket is attached to the second portion of the bracket while in other embodiments the first portion forms one body with the flexible cell culture pool. Furthermore, in some embodiments the second portion forms one body with the first portion.

In other embodiments the flexible cell culture pool is a flexible scaffold. In this case, the first side of each bracket comprises an adhesive layer, such as medical quality cyanoacrylate, for attaching the bracket to the flexible scaffold. A pair of screws may also be used to attach the flexible scaffold to a pair of brackets, respectively. Flexible scaffolds are more suitable when the cell culture that requires stimulation is a stem cell 3D culture. In some cases the flexible scaffold may be adhered to the recesses of the brackets and placed on top of a flexible cell culture pool used for the stem cell monolayer culture type.

In preferred embodiments the device is made of biocompatible material, such as polydimethylsiloxane (PDMS), and is sterilizable.

In another aspect of the invention the device also comprises a pair of electrodes. In some embodiments the electrodes are adapted to fit substantially in a recess of the brackets of the device. Furthermore, the pair of electrodes is attachable to a power source for at least electrically stimulating the cell culture or attachable to a controller for at least monitoring the impedance of the cell culture. In some embodiments each electrode comprises a platinum wire wrapped around a Polytetrafluoroethylene (PTFE) core.

In some embodiments, the device further comprises a second pair of electrodes attachable to a controller for monitoring the impedance of the cell culture when the first pair of electrodes is attached to the electrical stimulator.

In another aspect of the invention an apparatus is disclosed adapted to host at least one of the devices according to the first aspect of the invention. In some embodiments, the apparatus comprises at least one mechanical stimulator. The mechanical stimulator comprises a static magnet for attracting the first embedded ferromagnetic element of the hosted device in a non-moveable way and a free magnet for attracting the second embedded ferromagnetic element of the hosted device. The free magnet is attached to a moveable platform in a way that movement of the platform results in elongation of the flexible cell culture pool of the device to mechanically stimulate the cell culture.

In some embodiments the apparatus further comprises at least one electrical stimulator. In some embodiments the electrical stimulator comprises a set of two electrodes coupled to a pulse generator, wherein each of the two electrodes is placed in each of the recesses of a stimulation device. The pulse generator generates pulses that produce an electrical field between the electrodes to electrically stimulate the cell culture.

In other embodiments the electrical stimulator is adapted to be coupled to a pair of electrodes belonging to the stimulation device.

In a preferred embodiment the apparatus further comprises at least one aperture for hosting a Petri dish, wherein a stimulation device is placed inside the Petri dish, the Petri dish being placed between the first static magnet and the second free magnet, and wherein the diameter of the Petri dish is such as to allow elongation of the flexible cell culture pool.

In a preferred embodiment, as mentioned earlier, a third side of a bracket is curved so as to osculate with the internal side of a Petri dish.

In some embodiments the stimulation of the cell culture with the mechanical and the electrical stimulator is simultaneous.

In some embodiments the apparatus further comprises a plurality of apertures for hosting a plurality of Petri dishes. A device is placed in each Petri dish. The apparatus further includes at least a plurality of mechanical stimulators. Therefore a plurality of cell cultures may be simultaneously stimulated. In a preferred embodiment the apparatus includes six apertures for hosting a corresponding number of Petri dishes and devices. The apparatus may further include a plurality of electrical stimulators. In some embodiments the plurality of electrical stimulators is implemented with a single pulse generator and a multiplexer for supplying pulses to a plurality of electrode pairs. The electrode pairs may form part of the plurality of electrical simulators or may form part of the devices.

In yet another aspect of the invention a method is disclosed of stimulating a cell culture. In some embodiments the method comprises the steps of (i) selecting a stimulation mode, and (ii) stimulating mechanically the cell culture by extending the flexible cell culture pool.

In another embodiment the method further comprises the step of stimulating electrically the cell culture by creating an electrical field at the lateral sides of the flexible cell pool.

In a preferred embodiment the steps of stimulating mechanically and stimulating electrically are simultaneous.

The method may further comprise the step of sterilizing the device before placing it in a sterile Petri dish. This way the sterile barrier can be kept during the stimulation by effecting an external magnetic field to the whole set.

In general, most of the operational principles and advantages commented with respect to the embodiments of the device and the apparatus for stimulating electromechanically a cell culture, are also of application to the embodiments of the method for stimulating electromechanically a cell culture by said device and apparatus.

Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### BRIEF DESCRIPTION OF THE DRAWINGS

Particular embodiments of the present invention will be described in the following by way of non-limiting examples, with reference to the appended drawings, in which:

FIG. 1 is a perspective view of a device for stimulation of cell cultures, according to an exemplary embodiment.

FIG. 2 is a schematic representation of the device of FIG. 1 including the electrodes, according to another embodiment of the invention.

FIG. 3 is a schematic representation of an apparatus for electrical and mechanical stimulation of a single cell culture, according to an embodiment of the invention.

FIG. 4 is a schematic representation of an apparatus for electrical and mechanical stimulation of a plurality of cell cultures, according to an embodiment of the invention.

FIG. 5 is a flow diagram of a method of stimulation of cell cultures, according to an exemplary embodiment.

### DETAILED DESCRIPTION OF EMBODIMENTS OF THE INVENTION

In the following descriptions, numerous specific details are set forth in order to provide a thorough understanding of the present invention. It will be understood, however, by one skilled in the art, that the present invention may be practiced without some or all of these specific details. In other instances, well known elements have not been described in detail in order not to unnecessarily obscure the description of the present invention.

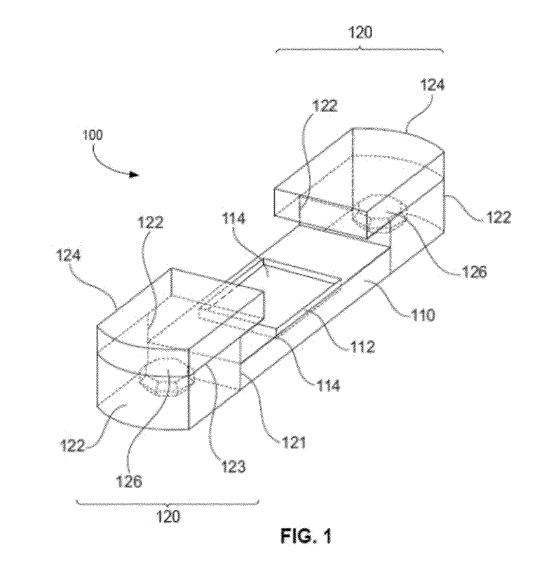

FIG. 1 is a perspective view of a device (100) for stimulation of cell cultures, according to an exemplary embodiment. Device 100 has a main body of a substantially rectangular shape. It comprises of a rectangular flexible cell culture pool 110 and two brackets (120) one placed diametrically opposite to the other. Flexible cell culture pool 110 comprises flexible structure 112 encompassing flexible substrate 114. Flexible structure 112 is arranged around the perimeter of flexible substrate 114 and is slightly elevated with respect to the flexible substrate so that the flexible substrate and the flexible structure form the flexible cell culture pool 110 that is adapted to accommodate a cell culture. Now, each of brackets 120 includes first portion 122 extending sideways to flexible cell culture pool 110 and is slightly elevated relative to the flexible cell culture pool. First portion 122 is adapted to accommodate embedded ferromagnetic element 126. Although any ferromagnetic material may be used for ferromagnetic element 126, a magnet is preferable. Bracket 120 further includes second portion 124 adjacent to first portion 122 on its upper side (123) and extending beyond the area of first portion 122 towards the flexible central part area so as to form a recess between flexible structure 114 and second portion 124. First portion 122 may be attached or may form one body with second portion 124. Equally, first portion 122 may be attached to or may form one body with flexible cell culture pool 110. Device 100 may be made of any biocompatible material, such as PDMS. Furthermore, the material of device 100 is selected so as to be fully sterilizable. As the ferromagnetic elements are embedded in the brackets, the device is sterilizable irrespective of the material of the magnet. This is particularly important as the device may be fully sterilized prior to accommodating the cell culture or may be sold directly pre-sterilized for immediate use.

Device 100 has been designed to facilitate the exertion of mechanical and electrical stimulation to a cell culture that may be hosted in flexible cell culture pool 110. More specifically, the mechanical stimulation is effected by attaching an external static magnet to the first of the two embedded magnets 126 of device 100 and using an external free magnet to attract the second of the two embedded magnets 126 in order to stretch and elongate flexible central part 110. Electrical stimulation is effected by an electrical field that is created by a pair of electrodes placed in the recesses of device 100.

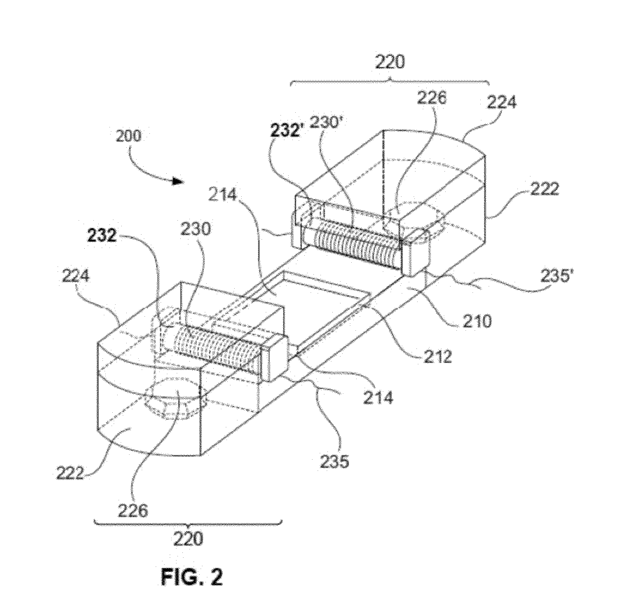

FIG. 2 is a schematic representation of the device of FIG. 1 including the electrodes, according to another embodiment of the invention. Device 200 includes flexible cell culture pool 210, brackets 220 including first portion 222 and second portion 224. Flexible cell culture pool 210 comprises flexible structure 212 encompassing flexible substrate 214. Flexible structure 212 is arranged around the perimeter of flexible substrate 214 and is slightly elevated with respect to the flexible substrate so that the flexible substrate and the flexible structure form the flexible cell culture pool 210 that is adapted to accommodate a cell culture. First portions 222 include first and second ferromagnetic elements 226. First and second electrodes 230, 230′ are provided in the recesses between the second portions 224 and the flexible cell culture pool 210. Electrodes 230, 230′ comprise of cores 232, 232′ and winding 235, 235′, respectively. The electrode winding should be constructed of a conducting, biocompatible and sterilizable material. Such material may be platinum, stainless steel or graphite. In case the conducting material has the form of a thin fibre, it may be winded around an insulating, biocompatible and sterilizable material such as Teflon to achieve structural integrity. As a result, the Teflon bar with the conducting fibre winded around it constitutes the electrode that can be placed in any of the recesses of the supporting device.

Electrodes 230, 230′ are coupled to a pulse generator (not shown). During operation, an electrical field is generated between the two electrodes and an electrical stimulation is effectuated to a cell culture that may reside in flexible cell culture pool 210. Apparently, it is possible to achieve mechanical, electrical, or simultaneous mechanical and electrical stimulation as the mechanical means for mechanical stimulation are independent to the electrical means for electrical stimulation. In our example the mechanical means are the external magnets that attract the embedded magnets to elongate flexible cell culture pool 210 and the electrical means are the electrodes that generate the electrical field.

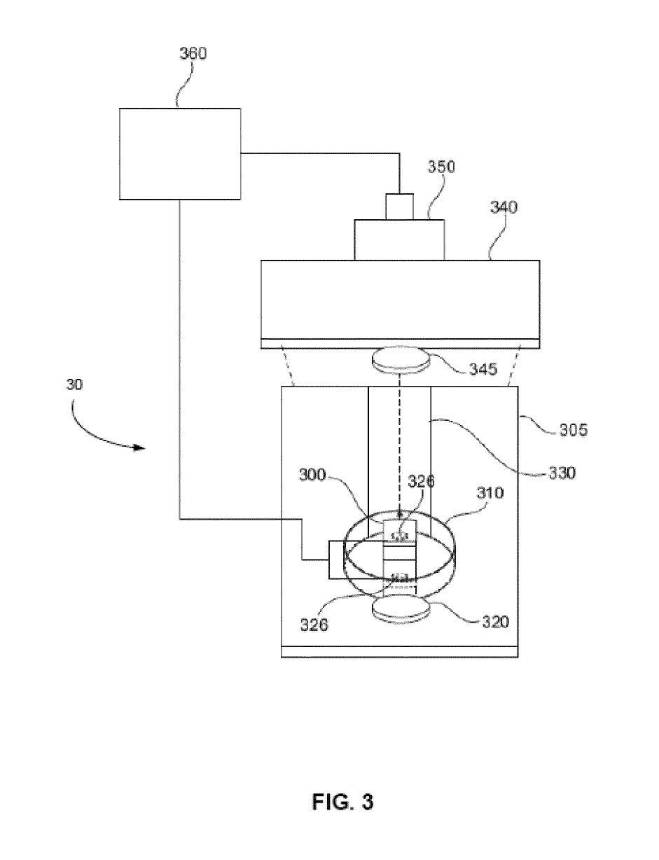

FIG. 3 is a schematic representation of an apparatus (30) for electrical and mechanical stimulation of a single cell culture, according to an embodiment of the invention. Apparatus 30 includes base 305 and platform 340. Base 305 includes an aperture sized to accommodate Petri dish 310. The Petri dish shall be used to host a device 300 similar to the device 100 or 200 described with reference to FIG. 1 or FIG. 2. Base 305 further includes static magnet 320 positioned at one edge of Petri dish 310. Along the virtual axis formed by static magnet 320 and the center of the Petri dish aperture, groove 330 is formed. As mentioned, apparatus 30 further comprises platform 340. Platform 340 is depicted vertical to base 305. Platform 340 is moveable with respect to base 305 along the axis of groove 330. Platform 340 includes free magnet 345 on one side facing base 305. Free magnet 345 is adapted to substantially fit into groove 330. Furthermore, both static magnet 320 and free magnet 345 have a magnetic field larger than the magnetic field of embedded magnet 326. Platform 340 includes mechanical arm 350 on its other side. Mechanical arm 350 is coupled to a motor (not shown) to effectuate movement of platform 340 along the axis of groove 330. Base 305 further comprises wiring for providing electricity to the electrodes (not shown) that will create the electric field for the electrical stimulation of a potential cell culture. Wiring of base 305 is coupled to power source 360. Power source 360 is also used to power the motor that is controlling movement of mechanical arm 350.

In a typical scenario, a device 300, substantially similar to the device described with reference to FIG. 1, hosts a cell culture in its flexible central part. The device is placed in a Petri dish. The Petri dish has a diameter slightly larger than the length of the device so that the device can be elongated within the Petri dish as a result of mechanical forces being applied to the device. Now, the Petri dish is placed in aperture 310 of base 305. One of the embedded magnets 326 of the device is attracted by static magnet 320. A pair of electrodes, residing in the recesses of device 300, is coupled to the wiring of base 305. The wiring of base 305 is coupled to a pulse generator inside power source 360. When the apparatus is in operation, an electrical field is applied to the cell culture and electrical stimulation is achieved. At the same time, arm 350 starts to move platform 340 in a direction towards the device 300. Consequently, free magnet 345 moves in a direction towards second embedded magnet 326 of device 300. When the force of the magnetic field of free magnet 345 exceeds a threshold force that relates to the elastic deformation of the flexible cell culture pool of device 300, the flexible cell culture pool starts to deform. During such deformation device 300 is elongated and mechanical stimulation is applied to the cell culture. This deformation continues until free magnet 345 is sufficiently removed from second embedded magnet 326 so that the force of its magnetic field is below the threshold force required to deform the flexible cell culture pool. It is apparent that since the magnetic field of static magnet 320 is larger than the magnetic field of free magnet 345, the first embedded magnet 326, and consequently the device 300, will not be separated from static magnet 320 when free magnet 345 attracts the second embedded magnet 326.

Apparatus 30 may include means for controlling the amplitude, frequency and duration of movement of arm 350 and consequently the amplitude, frequency and duration of the mechanical stimulation. Also, apparatus 300 may include means for controlling the amplitude, frequency and duration of the electric field applied to the cell culture for the electrical stimulation.

Apparatus 30 may also include an external electrical impedance measuring device for monitoring the evolution of the cell culture with the stimulation process in a non-destructive way.

As the electrical stimulation is achieved with the help of the two electrodes, the distance between the electrodes and the electrical potential applied to them determine the electric field applied to the cell culture placed in the central zone. In a preferred implementation the biocompatible device serves also for fastening the electrodes. For that purpose, the electrodes are placed in the recesses that are formed for that reason. The distance between the electrodes is, therefore, fixed by the recesses of the device. This ensures that the electric field applied to the cells does not change during the manipulation of the subject or during the simultaneous electromechanical stimulation. In the simultaneous stimulation, the electrical stimulation may be applied only in a predetermined position of the device (either stretched or relaxed, so that the deformation of the structure during the mechanical stimulation does not affect the electrical field due to the change of the distance between the electrodes.

In another scenario, electrical impedance spectroscopy can be used to perform monitoring of cell growth. This technique uses 4 (four) electrodes connected to an external electrical impedance measuring device and aligned in parallel in the zone in which cells grow, in the lower part of the stimulation device. An alternating current is applied by the external electrodes and the voltage drop is detected at the internal electrodes. The external electrodes may be the same as the ones used for electrical stimulation or may be two additional electrodes. The electrodes may be wires embedded in the flexible structure or they may be printed on the flexible structure or may be constituted by a conductive polymer embedded in the support. To avoid interference of the electrical and mechanical stimulation, the measure of impedance should be conducted in the dead time between stimuli. If carried out during mechanical stimulation, the measurement is influenced by the deformation. The measurement then does not serve to monitor the cells but rather to verify that the mechanical stimulation is applied on the stimulation device and to detect, for example, a possible rupture of the device.

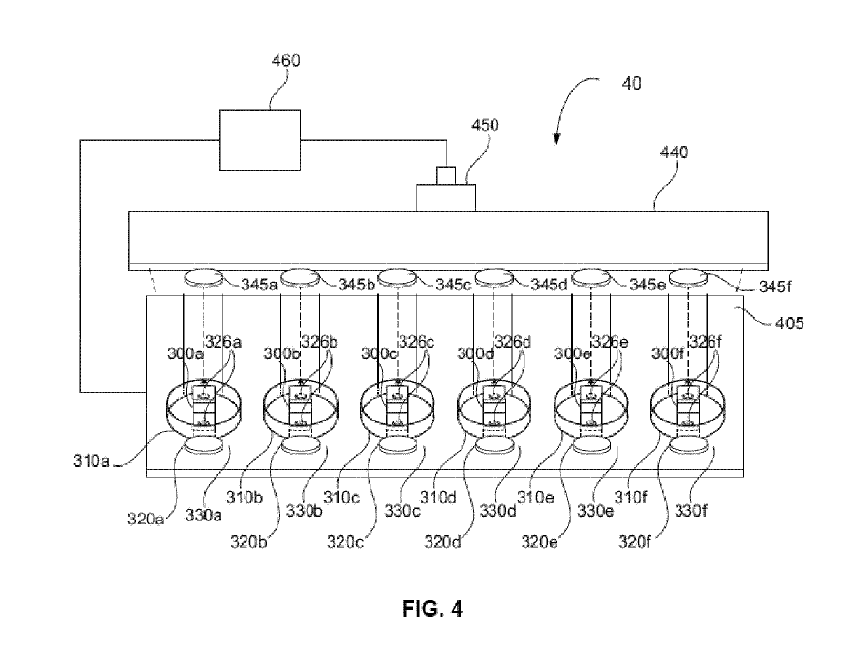

FIG. 4 is a schematic representation of an apparatus (40) for simultaneous mechanical stimulation or mechanical and electrical stimulation of a plurality of cell cultures, according to another embodiment of the invention. Apparatus 400 includes base 405 and platform 440. Base 405 includes a plurality of apertures sized to accommodate Petri dishes 310a . . . 310f. Although six apertures and corresponding Petri dishes are shown in this exemplary embodiment, one skilled in the art may appreciate that an apparatus similar to the apparatus of FIG. 4 may have any number of desired apertures. Each Petri dish shall be used to host a device similar to the device (100, 200) described with reference to FIG. 1 and FIG. 2. Base 405 further includes a plurality of static magnets 320a . . . 320f positioned at the edge of each Petri dish 310a . . . 310f, respectively. Along the virtual axis formed by each static magnet 320a . . . 320f and the center of each Petri dish aperture, a plurality of grooves are formed. As mentioned, apparatus 40 further comprises platform 440. Platform 440 is vertical to base 405. Platform 440 is moveable with respect to base 405. Platform 440 includes a plurality of free magnets 345a . . . 345f on one side facing base 405. Each of the free magnets 345a . . . 345f has a magnetic field larger than the magnetic field of each of the embedded magnets 326a . . . 326f. Platform 440 includes mechanical arm 450 on its other side. Mechanical arm 450 is coupled to a motor (not shown) to effectuate movement of platform 440. Base 405 further comprises wiring for distributing electricity to the electrodes (not shown) that will create the electric field for the electrical stimulation of the potential cell cultures. Wiring of base 405 is coupled to a pulse generator of power source 460. Power source 460 is also used to power the motor that is controlling movement of mechanical arm 450. The pulse generator may be internal or external to the power source 460.

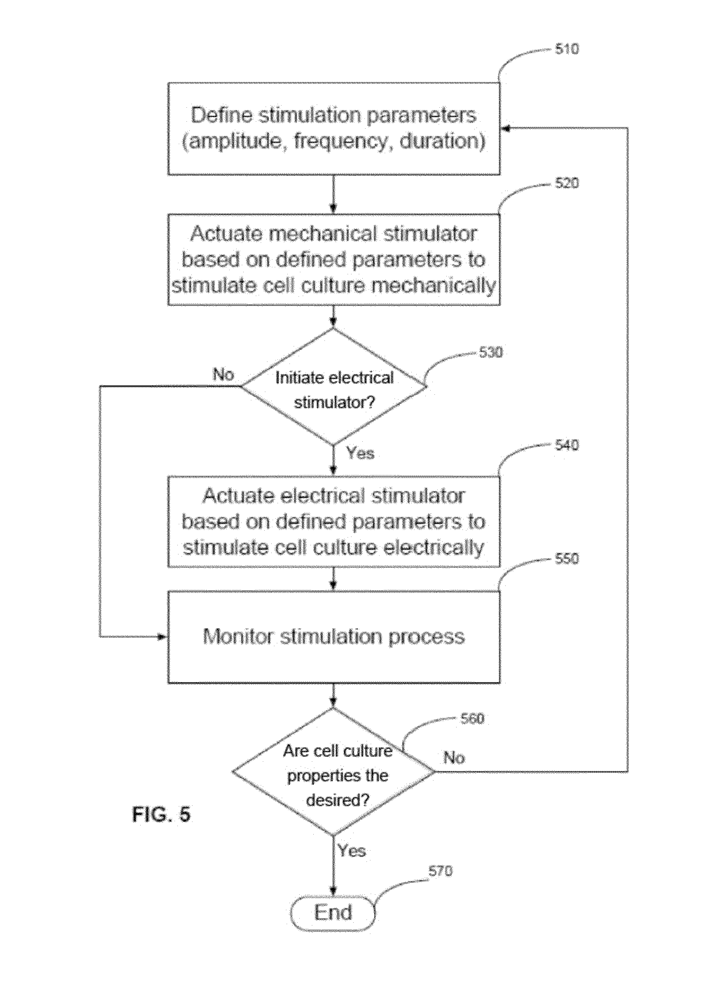

FIG. 5 is a flow diagram of a method of stimulation of cell cultures, according to an exemplary embodiment. a device substantially similar to that described with reference to FIG. 1 or FIG. 2 is placed in a electromechanical stimulator substantially similar to that described with reference to FIG. 3 or FIG. 4. In a first step 510, the stimulation parameters are defined. These may include one or more of an amplitude, a frequency and/or a duration of a stimulation. In the case of a mechanical stimulation, amplitude is the amplitude of the magnetic field which is proportional to the distance of a free magnet to a ferromagnetic element of the device. Frequency and duration refer to the frequency and duration of movement of the free magnet. In the case of the electrical stimulation, amplitude, frequency and duration refer to the amplitude, frequency and duration of the electric field between the electrodes of the device. Next, in step 520, the mechanical stimulator is actuated and the mechanical stimulation is initiated based on the defined parameters. The free magnet begins to approach the free embedded magnet of the device and, consequently, begins to attract the free later part of the device. As a result the flexible central part of the device starts to deform, and the mechanical stimulation of the cell culture begins. In step 530, a decision is made to whether the electrical stimulator should be initiated in order to have simultaneous electrical and mechanical stimulation. In case a simultaneous electromechanical stimulation is desired, then, in step 540, the electrical stimulator is actuated based on the defined parameters and the cell culture is stimulated electrically. Therefore, the cell culture is simultaneously electrically and mechanically stimulated. In step 550, a monitoring process monitors a value of at least a property of the cell culture and compares with a desired value. Preferably the monitoring process includes monitoring the value of the impedance of the cell culture. This monitoring may be continuous or at intervals. If, as seen in decision box 560, the value of the monitored property is substantially the desired one, or within a desired range, then the electromechanical stimulation continues with the same parameters till the end. If the value of the monitored property is not the desired one or is outside a desired range then the stimulation parameters are redefined and the stimulation continues with the new set of parameters until the stimulation process is considered successful.

Although only a number of particular embodiments and examples of the invention have been disclosed herein, it will be understood by those skilled in the art that other alternative embodiments and/or uses of the invention and obvious modifications and equivalents thereof are possible. Furthermore, the present invention covers all possible combinations of the particular embodiments described. Reference signs related to drawings and placed in parentheses in a claim, are solely for attempting to increase the intelligibility of the claim, and shall not be construed as limiting the scope of the claim. Thus, the scope of the present invention should not be limited by particular embodiments, but should be determined only by a fair reading of the claims that follow.

1. A bracket attachable to a flexible cell culture pool to form a stimulation device, the bracket comprising:
a first portion adapted to accommodate a ferromagnetic element, wherein a first side of the first portion of the bracket is attachable to a side of the flexible cell culture pool.

2. The bracket according to claim 1, wherein one or both:
the bracket further comprises a ferromagnetic element embedded in the first portion, and,
wherein one or both the ferromagnetic element and the embedded ferromagnetic element is a magnet.

3. (canceled)

4. The bracket according to claim 1, further comprising a second portion adjacent to a second side of the first portion, wherein the second side shares an edge with the first side, and wherein the area of the second portion extends beyond the area of the second side of the first portion.

5. The bracket according to claim 4, wherein one or more of:
the second portion is attached to the first portion;
the second portion forms one body with the first portion;
the second portion is made of biocompatible material; and,
the second portion is made of biocompatible material wherein the biocompatible material is polydimethylsiloxane (PDMS).

6. (canceled)

7. (canceled)

8. The bracket according to any of claim 1, wherein one or both:
the first portion is made of biocompatible material, and,
the first portion is made of biocompatible material wherein the biocompatible material is polydimethylsiloxane (PDMS).

9. (canceled)

10. The bracket according to claim 1, further comprising one or both:
an adhesive layer on the first side of the first portion, respectively, for attaching to the side of the flexible cell culture pool; or,
a third side of the first portion; wherein the third side is curved so that the bracket may osculate with an internal side surface of a Petri Dish.

11. (canceled)

12. The bracket according to claim 1, wherein the bracket is sterilizable.

13. A stimulation device comprising:
a flexible cell culture pool; and
at least a first bracket attached to the flexible cell culture pool,
the first bracket comprising:
a first portion adapted to accommodate a ferromagnetic element;
wherein the first side of the first portion of said at least first bracket is attached to a first side of the flexible cell culture pool.

14. The stimulation device according to claim 13, further comprising a second bracket having a first portion having a ferromagnetic element embedded therein, the second bracket attached to the flexible cell culture pool, so that the first side of the first portion of said second bracket is attached to a second side of the flexible cell culture pool so that the first bracket and the second bracket are placed one diametrically opposite to the other with respect to the flexible cell culture pool.

15. (canceled)

16. The stimulation device according to claim 13, wherein one or both:
the flexible cell culture pool is a flexible scaffold adapted to accommodate a cell culture that is a stem cell 3D culture, and,
the flexible cell culture pool comprises a flexible substrate encompassed by a flexible structure, wherein the flexible cell culture pool is adapted to accommodate a cell culture that is a stem cell monolayer culture and wherein the height of the flexible structure is greater than the height of the flexible substrate and lower than the height of the first side of each bracket so as to form a recess between the flexible structure and the second portion of each bracket, respectively.

17. The stimulation device according to claim 14, wherein the first portion of each of the first and second brackets forms one body with the flexible cell culture pool.

18. The stimulation device according to any of claim 14, further comprising one or both:
a first pair of electrodes each adapted to fit substantially in the recess of the first and the second brackets, respectively, wherein the first pair of electrodes is attachable to a power source for at least electrically stimulating the cell culture or attachable to a controller for at least monitoring the impedance of the cell culture; and,
a second pair of electrodes attachable to the controller for monitoring the impedance of the cell culture when the first pair of electrodes is attached to the electrical stimulator.

19. The stimulation device according to claim 18, wherein one or more of the electrodes comprises a platinum wire wrapped around a Polytetrafluoroethylene (PTFE) core.

20. (canceled)

21. An apparatus, adapted to host at least one stimulation device according to claim 14, having at least one mechanical stimulator and optionally also at least one electrical stimulator;
the at least one mechanical stimulator comprising:
a static magnet for attracting the embedded ferromagnetic element of a first bracket of the at least one stimulation device in a non-moveable way;
a moveable platform; and
a free magnet, attached to the moveable platform, for attracting the embedded ferromagnetic element of a second bracket of the at least one stimulation device in a moveable way, wherein the moveable platform moves in a way that the free magnet attracts the embedded ferromagnetic element of the second bracket and results in elongating the flexible cell culture pool of the stimulation device to mechanically stimulate the cell culture; and,
the at least one electrical stimulator adapted to be coupled to the at least one stimulation device to generate an electrical field to electrically stimulate the cell culture; and,
wherein when both the at least one mechanical stimulator and the optional at least one electrical stimulator are both included, the stimulation of the cell culture with the mechanical and the electrical stimulator is adapted to one or the other of occur asynchronously or simultaneously.

22. (canceled)

23. The apparatus according to claim 21, wherein the electrical stimulator comprises one or both of:
at least a pulse generator adapted to be coupled to a first pair of electrodes; and,
at least a pulse generator and a pair of electrodes coupled at one end to the pulse generator and to the other end adapted to be coupled to the stimulation device.

24. (canceled)

25. The apparatus according to claim 21, further comprising a controller for monitoring the impedance of the cell culture and for adapting at least one stimulation parameter when the impedance is not a desired one.

26. The apparatus according to claim 25, wherein the at least one stimulation parameter is a frequency, an amplitude or a duration of a stimulation.

27. The apparatus according to claim 21, further comprising at least one aperture for hosting a Petri dish between the first static magnet and the second free magnet, wherein the stimulation device is placed inside the Petri dish, and wherein the total length of the stimulation device is shorter than the diameter of the Petri dish to allow space for the elongation of the flexible cell culture pool.

28. (canceled)

29. (canceled)

30. The apparatus according to claim 21, wherein the apparatus is adapted to host a plurality of stimulation devices and wherein the apparatus further comprises one or both:
a plurality of mechanical stimulators to stimulate a plurality of cell cultures, respectively; and,
a plurality of electrical stimulators to stimulate a plurality of cell cultures, respectively.

31. A method of stimulating a cell culture comprising:
selecting a stimulation mode; and one or both:
stimulating mechanically the cell culture by extending the flexible cell culture pool of a stimulation device according to claim 13; and,
stimulating electrically the cell culture by generating an electrical field between at least two sides of the flexible cell culture pool; and,
wherein whenever both the steps of stimulating mechanically and stimulating electrically occur; these steps are one or the other of asynchronous and simultaneous.

32. (canceled)

33. (canceled)
